(19) United States
(12) Patent Application Publication
(10) Pub. No.: US 2010/0216120 A1
(43) Pub. Date: Aug. 26, 2010

(54) Rapid infectious virus assay

(76) Inventors: Candace Pert, Potomac, MD (US); Michael Ruff, Potomac, MD (US); Olivier Ducoudret, Kensington, MD (US); Lokesh Agrawal, Germantown, MD (US); Noel Baichoo, Catonsville, MD (US)

Correspondence Address: KILE GOEKJIAN REED & MCMANUS, 1200 NEW HAMPSHIRE AVE, NW, SUITE 570, WASHINGTON, DC, 20036, US

(21) Appl. No.: 12/711,070

(22) Filed: Feb. 23, 2010

Related U.S. Application Data
(60) Provisional application No. 61/154,754, filed on Feb. 23, 2009.

(51) Int. Cl. C12Q 1/70

(52) U.S. Cl. 435/5

(57) ABSTRACT

An assay to detect or quantify HIV infectious virus from clinically relevant cellular compartments, or reservoirs, in anti-retrovirally treated patients whose viral levels are low to undetectable is described. The method detects infectious virus in patients whose plasma viral loads are considered to be below the limit of current PCR based detection methods and thereby is more relevant for guiding treatment. A further advantage is that the method allows viral tropism to be directly determined in the presence of specific inhibitors of CCR5 or CXCR4. Drug sensitivity can also be directly determined without the need to laboriously recover patient virus by culture for extended time periods, a method that allows for viral selection or evolution, which is not desirable. Patient cells, like the blood mononuclear cells, or monocytes, are isolated and cultured in the presence of cytokines like CSF-1/M-CSF or GM-CSF. to promote their differentiation. Cells are activated with lectins, mitogenic antibodies, phorbol esters, Toll Receptor stimulation or inducers of NfKb or NFAT, followed by agents that induce viral release, like ATP or stimulation of autophagy with LiCl, spermidine, or rapamycin. A key aspect of the invention relates to the timing of the addition of these agents for optimal viral release. A further aspect of the invention relates to sensitive detection of released virus which can be accomplished by adding so-called reporter cells which are under control of the HIV TAT protein so that upon infection these cells synthesize proteins or enzymes that allow for the measurement of infectious particles.

This application claims priority to U.S. Provisional Application Ser. No. 61/154,754, filed Feb. 23, 2009, the contents of which are hereby incorporated by reference.

### BACKGROUND

No simple, reproducible, fast assay currently exists to measure infectious Human Immunodeficiency Virus (HIV) in patients. Direct assays of bodily fluids such as serum and blood plasma is difficult. It is estimated that defective HIV virus particles make up over 99.9% of the virus found in blood plasma. Defective particles may outnumber active, infective virus by ratios of 10,000 to 1, or more. Adding to difficulties in measurement, HIV levels can appear to drop to zero, for example, in patients undergoing Highly Active Anti-Retroviral Treatment (HAART) only to reemerge after HAART is stopped. Patients who are completely suppressed—that is, patients who have no detectable virus in their blood plasma, nevertheless still have active HIV lingering in cellular reservoirs such as CD4 lymphocytes and monocytes. Tissue compartments comprising these persistently infected or latent, cells, sometimes called “viral reservoirs”, are the source, and/or is in equilibrium with the source, of virus which re-infects the body after HAART is stopped. Thus, the direct measure of infectious virus in addition to other surrogate markers is needed to establish the requirement and effectiveness of any particular antiviral treatment.

### PRIOR ART METHODS

The re-infection is currently detected by a rise in blood plasma levels of viral RNA for the HIV protein gag (p24). The gag-viral RNA levels are measured using the Roche Amplicor PCR method, currently the only approved method of monitoring patient viral levels. However polymerase chain reactions, while very sensitive, do not distinguish infectious virus from defective, non-infectious particles, and are not as sensitive as methods based on recovery of infectious virus.

The persistance of HIV after therapy is the main obstacle to a cure. Human immunodeficiency virus (HIV) causes a chronic and eventually fatal disease in humans. The time course of the disease is slow and involves the continuing virus replication and spread in the face of a specific antiviral response by all arms of the immune system.

After an early and rapid rise in blood virus a ‘set-point’ level of lower chronic viremia is established. When treated, plasma virus levels become undetectable in the majority of patients. Tissue infection and helper T cell destruction however is widespread and a situation of latent and chronic active virus production occurs. Latency refers to a state in which HIV has become stably integrated into the cells DNA, however no virus or viral proteins are produced. The threat of latency is that virus may become re-activated, most commonly as part of normal immune defenses to infection or injury. In the latent state cells avoid immune attack and in long-lived cells like the memory T cells, last for a lifetime. Persistence refers to a chronic condition in which virus is produced at low levels, even with therapy, and slowly spreads even as infected cells may die or be replaced. Both processes lead to life-time infection. (1-3). The susceptibility of latently infected cell populations to HIV treatments, their proliferative capacity, and ability to produce infectious virus subsequent to alterations in cellular physiology are critical issues which determine the contribution of these cells to viral persistence.

Patients are assumed to need treatment over the entire course of their disease due to the rebound in viremia upon cessation of their antiviral medicines (4-6). Any attempt to cure HIV infection must suppress the rebound of virus from the treatment resistant, persistently infected clinically relevant cellular compartments (CRCC), sometimes called “viral reservoirs” or “sanctuaries”.

Several problems remain to be overcome in order to eliminate CRCC's. Firstly, the clinically relevant cellular compartments for persistent viremia remain to be identified, and then cells must be able to be recovered, and tested for infectious virus in a simple, rapid and sensitive test in order to evaluate treatments and their efficacy in reducing infectious viral load in patients.

### Low Level Viremia in Clinically Relevant Cellular Compartments.

Once a person becomes infected progression to the acquired immunodeficiency syndrome (AIDS) correlates with the reappearance of circulating viral proteins. Low level viremia is a problem during the long middle period of infection due to the continuing immune activation and ensuing functional immunological anergy and deficiencies. There continues to be extensive viral infiltration in brain, gut, spleen, lymph nodes, and lungs, which leads to specific pathologies, eg Neuro-AIDS and memory losses (7, 8). Infection progresses even though at any given time in the lymphoid tissues integrated HIV-1 DNA is present in only a minute fraction of the susceptible populations (9). Chronically infected tissues and cells include T cells, monocytes (10, 11), macrophages (12-14), gut; (15, 16), brain (17, 18).

There are several potential cellular and anatomical reservoirs for HIV-1 that may contribute to long-term persistence of HIV-1. These include infected cells in the brain, gut, bone marrow, lungs, and genital tract. Most attention and focus has been on the reservoir of latently infected resting memory CD4(+) T cells which have a long life span and have the ability to reactivate upon encounter with an antigen or other stimulation and that harbor latent HIV-proviral DNA. Since replication-competent virus can be routinely recovered from resting CD4+ T lymphocytes in patients successfully treated with HAART for up to 7 years (3) a leading hypothesis is that quiescent CD4+ T lymphocytes carrying proviral DNA provide a reservoir for re-bounding HIV in patients on highly active antiretroviral therapy (HAART) (19)

The most important reservoirs however are those that lead to re-emergence of virus after interruption of HAART therapy (2, 4). Virus may be recovered, in vitro, by ‘heroic’ use of large numbers of patient cells, extensive cellular activation with mitogenic antibodies or lectins, and culture for long time periods, with cell replenishment, but these methods and approaches do not capture the pool of virus that arises in patients with cessation of therapies (2, 4). Thus, despite prevalent opinion, viral rebound after discontinuation of therapy may not arise from the latent T cells.

The half-life of this latent reservoir is extremely long (44 months). At this rate, eradication of this reservoir would require over 60 years of treatment (20). The extraordinary stability of the reservoir may reflect gradual reseeding by a very low level of ongoing viral replication from other CRCC, most likely including hematopoietic progenitors arsing in bone marrow, liver, or other sites.

Thus, in the majority of patients after discontinuation of highly active anti-retroviral therapy (HAART), the rebounding plasma virus was genetically distinct from both the cell-associated HIV RNA and the replication-competent virus within the detectable pool of latently infected, resting CD4+ T cells (4, 20). Thus the rapid emergence of plasma viremia after cessation of HAART does not seem to be due to the release of virus stored in latently-infected cells that have become activated, but probably results from residual replication of still-activated cells.

Monocytes also contribute to HIV-1 persistence and comprise a very important CRCC. Bone marrow derived cells, that are monocyte-like, adherent, that express chemokine receptors, especially CCR5, are proposed to be a CRCC. Although monocytes are a minor HIV DNA reservoir, these cells circulate in the blood up to 3 days and then migrate to various tissues where they differentiate in to macrophages and their life span could vary between a few days to several months. The circulating monocytes have low level active virus production with no immune response generated as they have limited expression of viral proteins on their surface. Once entering tissues however and differentiating into macrophages, dendritic cells, microglia etc. they can infect T cells or release viral proteins that cause death or functional alterations of neighboring cells.

Thus cells of macrophage lineage, including monocyte subsets within the blood, play a role in HIV-1 persistence (21,10). Evidence of sequence evolution in blood monocytes, in comparison to resting CD4+ T cells, demonstrates their distinct contribution to plasma viremia. There is evidence to suggest that a specific monocyte subset, of CD14loCD16hi phenotype, is more susceptible to HIV-1 infection than the majority of blood monocytes. (22). Trafficking of monocytes through various tissues following their emigration from the bloodstream allows these cells to differentiate into tissue macrophages, or potentially to egress from the tissues as migratory dendritic cells. The circulating infected monocytes (21) likely derived from the true clinically relevant cellular compartment in the bone marrow. There monocyte precursors first arise, become infected, enter the blood, and then in a few days move into the tissues to spread viremia. Because the monocytes only circulate for a few days, the ability to easily capture these continually regenerating circulating cells provides the basis for a measurement of the CRCC. As this compartment is eliminated the burden of circulating monocytes can be easily measured by collecting blood, a simple procedure. The size of the infected lymph-node monocyte-macrophage pool has been estimated to be 50 in 10⁶ macrophages (1). The cells of monocyte-macrophage lineage also carry a wide range of HIV strains (2) and consist of up to 10% of productively infected cells in early stages of the disease, and may increase significantly in later stages of disease when CD4 T cells are. Several reports have documented that infectious virus could be detected in circulating monocytes from patients on HAART for prolonged periods of time (3, 4). The virus normally reactivates following appropriate stimulation. HIV reactivation in promonocytic cell lines has also been shown following cell to cell contact with CD+T cells (5). Monocyte-derived macrophages have also shown to spread the virus to CD+T cells by fusing with autologous and heterologous CD+T cells (6)

Because infection is established in the body by CCR5 using strains of HIV the most relevant source of persistent virus that rebounds after HAART cessation is a blood derived cell, that originates from a persistently infected cell in the bone marrow, that is hemopoietic in nature, probably monocyte-like, and which expresses CD4 and CCR5. Monitoring these cells, by the methods described herein, provides critical information to guide the treatment and management of patients who wish to discontinue antiviral therapy for a period of time. A rapid assessment is needed to advise clinicians who may elect to re-start therapy if an early viral rebound is detected. In the same manner, sustained suppression of this CRCC could justify additional time off therapy. Patients wish to interrupt their treatments due to the long-term side effects of current antiviral medicines.

A second problem, after the identification of the CRCCs, is the lack of a simple and rapid method to detect, measure, and quantify infectious cellular virus. The most sensitive method of virus detection involves recovery (often called ‘rescue’) of infectious HIV. Replication-competent cells with integrated provirus are detected at less than 1 in 10(6)-10(7) cells, ie, extremely rarely. These replication competent cells are only detected by heroic measures that involve large numbers of patients cells, cultured for extended periods of time, with constant replenishment of the culture media and addition of fresh cells. This type of virus ‘rescue’ is in general use (1, 9, 17, 23), however it is costly, laborious, and time consumptive, taking up to three weeks to perform. This method is not amenable to large scale testing of clinical samples, nor can it provide meaningful information to clinicians in a timely fashion to inform treatment decisions, rather its use is in small samples in a laboratory setting.

Other sensitive methods of detecting cellular HIV, such as PCR methods, do not detect infectious virus as the most prevalent form of HIV-1 DNA in resting and activated CD4+ T cells is a full-length, linear, unintegrated form that is not replication competent. (9).

In the plasma, PCR methods, which are in wide use and are the basis for the only approved clinical test of viral load (Roche Amplicor test) measure only viral genomic material which is widely understood to mostly reflect non-infectious, so-called ‘defective’ viral particles (24). This test is not sensitive enough to detect a single particle of infectious virus. This degree of sensitivity is desired as it is recognized that a single virus can emerge to re-infect the entire body.

### BRIEF SUMMARY OF THE INVENTION

The inventors have developed a new method of measuring HIV infection levels. In one embodiment, samples to be assayed are derived from freshly isolated peripheral blood monocytic cells (PBMC). In another embodiment, the PBMCs can be depleted of their CD8 lymphocytes. In other embodiments samples are prepared from any suspected HIV cellular reservoir, derived either from a patient's tissues or blood.

Monocytes are the major HIV expressing cell in the blood of treated patients with suppressed viral load. Monocyte production of virus is enhanced by contact with activated T cells (7) and a preferred method involves mixed culture of monocytes and T cells.

To maximize HIV production, the samples are placed in reaction vessels supplemented with fibronectin. It has also been shown previously that fibronectin binds to gp120 and pretreating HIV-1 with fibronectin increases the infectivity of HIV-1, when a low concentration of the virus is present (8) This increases HIV production by promoting adherence of the cells to the reaction vessel surface and presentation of virus to entry receptors. The sample cells are then incubated for a sufficient time to allow for cell adherence as well as syncytia formation, cellular morphological modifications and bridging of cell surface and virions that further promote HIV stability and production.

HIV-infected monocytes, even if made transcriptionally active, may not release their viruses. In fact it is understood that monocytes tend to accumulate transcribed virus intracellularly. In order to measure this virus agents that cause virus release are helpful. Thus added ATP (9), or chemically induced autophagocytosis, (10), by LiCl, spermidine, or rapamycin can be used to elicit virus release. An alternative method of enhancing virus release is to culture the monocytes under non-adherent conditions, for example in Teflon dishes, without activation (11).

To maximize the assay's sensitivity, the sample, either patients' cells, or the culture supernatants derived therefrom, is combined with any of several “reporter cells.” These reporter cells are genetically engineered so that when infected by HIV the cells give off a readily detectable signal. Any reporter cell is suitable for use in the method. One example of a suitable reporter cell is a TZM cell. TZM cells are HeLa-cell derivatives that express high levels of CD4 and both co-receptors CXCR4 and CCR5. TZMs are derived from a parental cell line (JC.53) which stably expresses large amounts of CD4 and CCR5. The cell line was generated from JC.53 cells by introducing separate integrated copies of the luciferase and β-galactosidase genes under control of the HIV-1 promoter. HIV infection results in the induction of luciferase and β-galactosidase allowing easy detection of infection and titration. The TZM-bl cell line is highly sensitive to infection with diverse isolates of HIV-1

Other suitable reporter cells use reporter genes fused to promoters from genes known to be activated upon HIV infection. An example of this type of reporter cell is a GHOST cell (12).

The sample and the reporter cells are combined in a suitable medium. Within three to seven days a quantitative readout of viral levels per cell can be obtained.

It is novel to use reporter cells directly with cells containing the HIV cellular reservoir.

In still other embodiments, agents can be added which stimulate the cells to release or supply more virus such as the phorbol esters PMA and prostratin or mitogens like PHA or ConA. Stimulation of autophagy can also promote virus release (10) These conditions will further enhance the sensitivity of the assay by inducing production of more virus into the culture medium. As disclosed herein, CD8 cells can be removed from the PBMC population before addition of the reporter cells as they are not a source of virus and may in fact interfere with virus detection. This should also increase assay sensitivity by removing chemokines produced by the CD8 cells which inhibits virus infection. The invention also includes any other methods of stimulating viral release or reducing inhibitors to viral infection.

The invention has broad application to non-invasively measure and monitor patient viral levels to plan treatment regimens. An added advantage of this method is the fact that it will only measure active, infectious virus not “defective particles” found in blood plasma and thus be more accurate and predictive of disease progression. A further advantage of the invention is its ability to determine drug sensitivity directly, without cultivation or rescue of virus under long-term culture conditions which can lead to virus evolution or divergence from the actual clinical sample. The receptor-usage or tropism of primary patient viral isolates can also be determined at the same time by including inhibitors of specific co-receptors like CCR5 or CXCR4. This is important because patient treatments are based on the predominant co-receptor usage.

### BRIEF DESCRIPTION OF THE FIGURES

FIG. 1: ATP alone does not trigger the release of the virus from CD-depleted monocytes: 200 k CD8-depleted PBMCs were plated on to a 96-well for 3 hrs followed by washings using RPMI 10%. ATP (1 mM) was then added in a volume of 100 ul for a 3 hrs at 37° C. TZMs (10K) were then added on to the wells in 100 ul in RPMI 10%. After 3 days later, the supernatants were removed and steady-glo reagent was added on to the TZMs and read on a Spectramax.

FIG. 2: Combination of ATP and fibronectin triggers the release of the virus from CD-depleted monocytes (150 K) 5 day assay (Format #1): TZMs were plated on to a 96-well at 5 k in RPMI 10%. The plates were then coated with fibronectin and BSA. 150 k CD8-depleted PBMCs were added on to a 96-well for 3 hrs followed by washings using RPMI 10%. ATP (1 mM) was then added in a volume of 100 ul for a 3 hrs at 37° C. TZMs (15K) were then added on to the wells the next day. 5 days later after launch, the supernatants were removed and steady-glo reagent was added on to the TZMs and read on a Spectramax.

FIG. 3: Combination of ATP and fibronectin triggers the release of the virus from CD-depleted monocytes (100K) 4-day assay (Format #2): 100 k CD8-depleted PBMCs were added on to a 96-well (coated with fibronectin and BSA) for O/N followed by washings using RPMI 10%. ATP (1 mM) was then added in a volume of 100 ul for a 3 hrs at 37° C. TZMs (10K) were then added on to the wells. 4 days later after launch, the supernatants were removed and steady-glo reagent was added on to the TZMs and read on a Spectramax.

FIG. 4: Combination of ATP and fibronectin triggers the release of the virus from CD-depleted monocytes (100K) 3-day assay (Format #3): 100 k CD8-depleted PBMCs were added on to a 96-well (coated with fibronectin and BSA) for O/N followed by washings using RPMI 10%. ATP (1 mM) was then added in a volume of 100 ul for a 3 hrs at 37° C. TZMs (10K) were then added on to the wells. 3 days later after launch, the supernatants were removed and steady-glo reagent was added on to the TZMs and read on a Spectramax.

FIG. 5: Combination of ATP and fibronectin triggers the release of the virus from CD-depleted monocytes (225 k) 4-day assay (Format #4): 100 k CD8-depleted PBMCs were added on to a 48-well (coated with fibronectin and BSA) for O/N followed by washings using RPMI 10%. ATP (1 mM) was then added in a volume of 100 ul for a 3 hrs at 37° C. TZMs (20K) were then added on to the wells. 3 days later after launch, the supernatants were removed and steady-glo reagent was added on to the TZMs and read on a Spectramax.

### DETAILED DESCRIPTION

Detection of infectious HIV particles from patients' Peripheral Blood Mononuclear Cells (PBMC) using classical co-culture methods is time-consuming and focuses on CD4 cells. The inventors propose a 4 day cell-based approach capable of detecting virus in suppressed patients. Rather than lymphocytes, this method monitors the putative clinically-relevant cellular compartment (CRCC) believed to comprise CD14-positive monocytes. In conjunction with this method, real-time PCR (RT-PCR) and flow cytometry assays have been developed to monitor CRCC infection in suppressed patients for mDAPTA (13) in phase II clinical studies.

In a preferred embodiment, CD8-depleted PBMC were isolated from HIV-negative donors and HIV patients with VL<50 copies/ml using Dynal beads. Peripheral monocytes (CD14) were used in a rapid infectious assay using TZM-bl coculture technique. The inventors compare this shorter infection assay to classical method of PBMC activation with anti CD3/CD28 antibodies. Major CCR5 blockers were addressed. Presence of infectious virus was also investigated using FACS and rtPCR.

As will be shown below, the inventors detected infectious HIV in all studied patients' (VL<50) purified CD14-monocytes using a Rapid Infectious Viral Assay (RIVA). Virus rescue in RIVA format was comparable to the traditional rescue methods. The virus released was found to be sensitive to CCR5 inhibitors. Our cellular assay confirms that live HIV particles are released by the monocytes, even in absence of stimulating drug. The inventors also found that monocytes constituted a significant source of virus as assessed by PCR and FACS analysis.

The inventors also determined that the monocytic compartment is a significant source of the viral burden. Furthermore a 4 day cellular assay confirms that live HIV particles are released by the monocytes. Since the assay didn't detect any significant increase of infectivity in presence of activators, the inventors propose that this assay provides a direct measurement of the cellular viral burden and not merely the ability to generate de novo infectious particles. The method avoids the confounding factors of evolution/selection in long term cultures.

Other agents believed to increase assay performance are factors that promote the progress of sample cells down their developmental pathway. The undifferentiated monocytes are not highly transitionally active and do not release virus efficiently (14). Addition of the differentiation factors M-CSF or GM-CSF to accelerate formation of macrophages in culture is desirable. To enhance virus production, activation of differentiated monocytes by stimulation of Toll receptors, for example by addition of endotoxin or other bacterial products, or stimulation using cytokines that activate NfKb or NFAT (15), which are regulators of virus transcription are useful. This can be accomplished by adding cytokines like TNFa, IL-1, IL-6, lectins, or activating antibodies, like anti-CD3, anti-CD28.

The primary use of the method is to increase the sensitivity, precision and accuracy of HIV detection assays. The method described herein will reduce the time in which a patient must wait for the assay results as well as reduce the incidence of false positives.

The inventors envision are many other uses. The assays can be used to guide treatment. For example, the goal of a typical HART treatment is to reduce CRCC HIV titer to zero. In a preferred embodiment, a patient can be taken off HART when the CRCC HIV titer reaches zero. Thus, the assays disclosed herein will be useful to determine the stopping point of certain HIV therapies.

There are other uses for the disclosed assays. HIV patients often are involved in clinical trails having side-by-side comparisons of different drug treatments. The disclosed assays can be used to closely monitor the relative effects of the different drugs. Thus, the methods can be used in evaluating new HIV drugs.

In still another use, drug resistance can be monitored. By monitoring a patients response over time to a drug treatment regimen, the assay can be used to detect a rise in HIV titers that signal the onset of drug resistance. Moreover, in the case of many drugs, it can be determined that the drug acts by blocking HIV binding to a specific cognate entry receptor. In these cases, the assay suggests the phenotype of drug resistance. Either HIV is somehow has modified so that it out competes the drug for receptor binding or the HIV has evolved so that a different entry receptor is used.

In the case of the later event, the assay can be used to verify that a different entry receptor is being used. The assay should show an increase in sensitivity of the HIV to a drug known to bind to the new entry receptor.

In yet another use, the assay can determine the specific phenotype of an emerging HIV drug resistant strain. For example, HIV drug resistance can be the result of a mutation in the virus's CCR5 or CXCR4 binding function. Alternatively, HIV can evolve to use both receptors. The assay can be used to distinguish between CCR5 and CXCR4 binding. If the HIV uses one receptor, the assay will reveal the HIV is sensitive to drugs known to block HIV binding to that receptor, but resistant to drugs known to block the other receptor. If the HIV uses both receptors, then both drugs should be required to block infection.

### Example 1

### The Importance of ATP for Viral Release

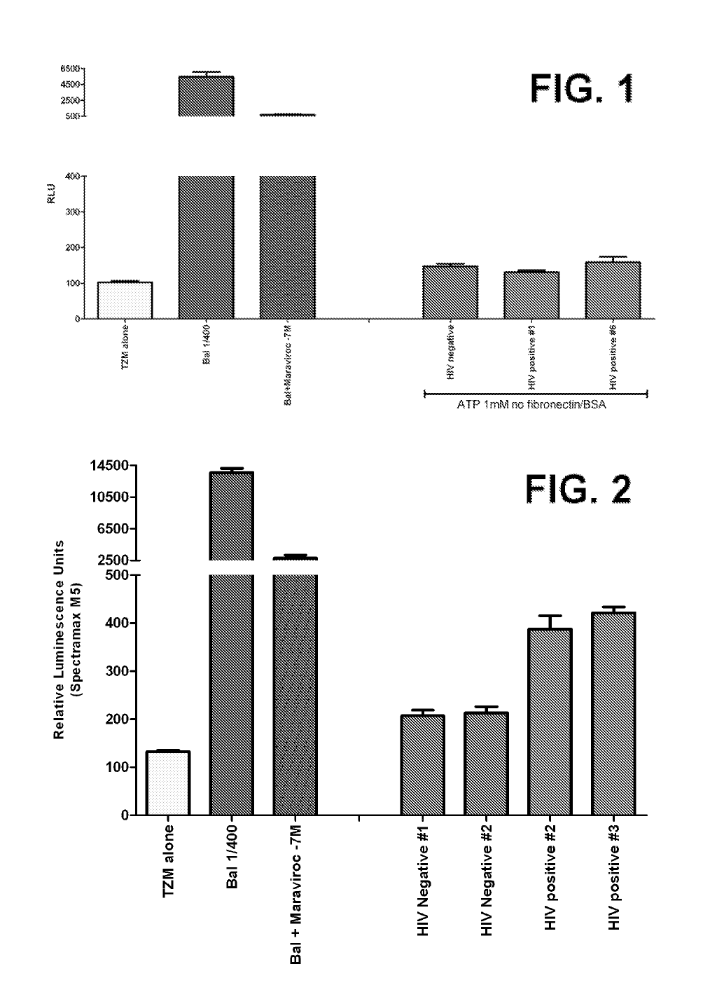

As shown in FIG. 1, ATP by itself does not fully promote release of the virus from the sample cells. The inventors tested the ability of ATP (1-5 mM) to release infectious virus from monocytes in absence of fibronectin coating on to the tissue culture wells. The relative light units which is a measure of infectivity of HIV on TZMs was measured and was found to be similar in both HIV-positive and negative individuals. The results demonstrate that ATP by itself is incapable of triggering release of viral particles from monocytes (FIG. 1). Further differentiation and/or activation is useful to enhance virus release. Ba-L which is used as a positive control shows good infectivity on TZMs.

### Example 2

### Detection of HIV in Monocytes Treated with ATP

FIG. 2 illustrates a test of the ability of ATP (1 mM) to release infectious virus from monocytes in presence of fibronectin coating on to the tissue culture wells. The HIV infectivity was measured by measuring the RLU on a Spectramax M5 plate readert. There was significantly higher virus detected in monocytes cultured from HIV positive patients when compared to HIV negative donor. Thus ATP in presence of fibronectin is capable of triggering release of viral particles from monocytes (FIG. 2). Ba-L which is used as a positive control shows good infectivity on TZMs.

### Example 3

### Overnight Incubation

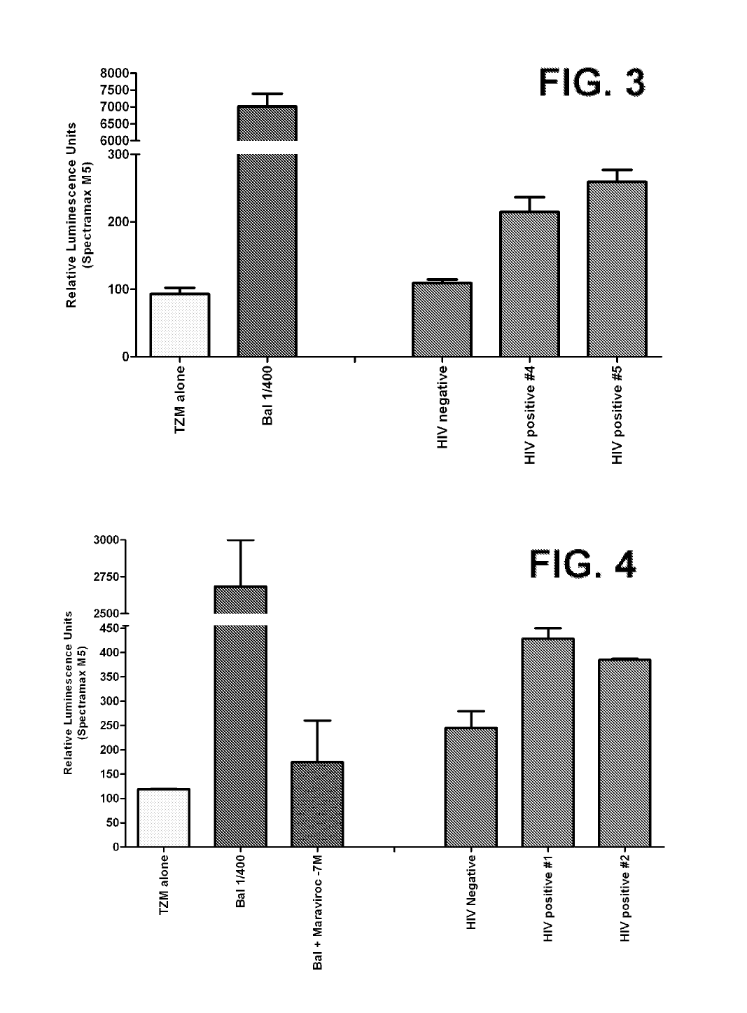

FIG. 3 illustrates a test of the effect of overnight/N plating of monocytes to increase the number of monocytes sticking to the plate and thus the RIVA signal. Although there was a significant increase in virus detected in monocytes cultured from HIV positive patients as compared to HIV negative donor (FIG. 3), but the virus release was found to be similar as compared to that obtained using 3 hrs adherence (FIG. 2).

### Example 4

### Use of 100K Sample Cells

We then looked at the possibility if we could shorten the length of the assay to 3 days using 100K cells as is illustrated in FIG. 4. There was a significant increase in virus detected in monocytes cultured from HIV positive patients as compared to HIV negative donor (FIG. 4) and the virus release was found to be similar as compared to that obtained using 150 k CD8-depleted PBMCs (FIG. 2).

### Example 5

### Use of 225 K Sample Cells in a 48 Well Microtiter Plate Format

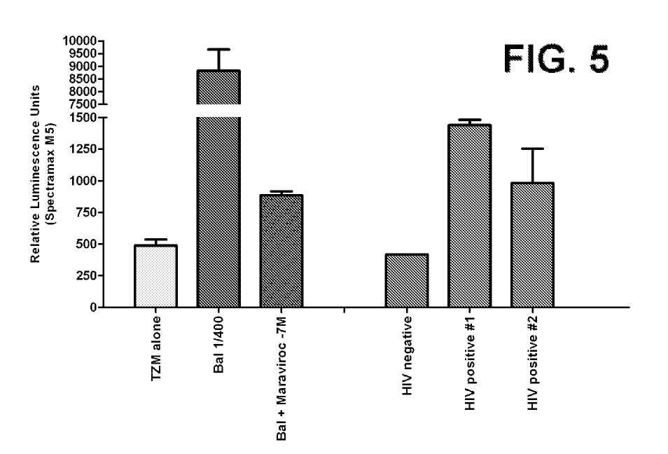

We also looked at the possibility of using a higher cell number (225K) of CD-depleted monocytes in a 48-well format as illustrated in FIG. 5. We found that there was a significant increase in virus detected in monocytes cultured from HIV positive patients as compared to HIV negative donor (FIG. 5) and the virus release was found to be significantly higher as compared to that obtained using 100-150 k CD8-depleted PBMCs (FIG. 2, 3, 4).

### Example 6

### A Preferred Protocol

The following is a preferred protocol for the assay:

1. A 96 well plate (a 48 well can be used) is coated with 100 ul RPMI containing fibronectin (50 ug/ml) in BSA (0.5%) for 30 minutes at room temperature.

2. CD8-depleted PBMC's which have been frozen at 2 million cells per ml and stored at −80 C in 22% FBS and 10% DMSO are thawed quickly and about 100 ul containing 200 k cells are added to the wells immediately

3. Let cells of interest attach for 2 hours (longer even overnight up to 20 hrs can be used)

4. Remove all the media which contains non-adherent cells and wash with 200 ul of RPMI with 10% FBS, pipetting gently, removing this wash media. The adherent monocytes remain.

5. Add 100 ul of RPMI containing 10% FBS (10% human AB serum can be used=containing 1 mM or more ATP up to 5 mM to release HIV from adherent cells, then incubate at 37 degrees C. for 3 hours

6 Add 10 k of TZM bl reporter cells per well in 100 ul of RPMI-10% FBS and incubate for 3 days. 20 k TZMs could be added if a 48 well format is used.

7. Finally quantitate how much viral infection has occurred by reading luciferase production using Steady Glow reagent (Promega) and a luminometer/spectrophotometer.

8. In order to characterize type of infectious virus, perform each patient's baseline determination in triplicate and include maraviroc (16) and DAPTA (Dala1-peptide T-amide) (13) triplicates to determine CCR5 tropic virus and AMD 3100 (17) to determine CXCR4 tropic virus.

### LITERATURE CITED

1. Chun, T. W., L. Carruth, D. Finzi, X. Shen, J. A. DiGiuseppe, H. Taylor, M. Hermankova, K. Chadwick, J. Margolick, T. C. Quinn, Y. H. Kuo, R. Brookmeyer, M. A. Zeiger, P. Barditch-Crovo, and R. F. Siliciano. 1997. Quantification of latent tissue reservoirs and total body viral load in HIV-1 infection. Nature. 387:183-188.
2. Xu, Y., H. Zhu, C. K. Wilcox, A. van't Wout, T. Andrus, N. Llewellyn, L. Stamatatos, J. I. Mullins, L. Corey, and T. Zhu. 2008. Blood monocytes harbor HIV type 1 strains with diversified phenotypes including macrophage-specific CCR5 virus. J Infect Dis. 197:309-318.
3. Lambotte, O., Y. Taoufik, M. G. de Goer, C. Wallon, C. Goujard, and J. F. Delfraissy. 2000. Detection of infectious HIV in circulating monocytes from patients on prolonged highly active antiretroviral therapy. J Acquir Immune Defic Syndr. 23:114-119.
4. Zhu, T., D. Muthui, S. Holte, D. Nickle, F. Feng, S. Brodie, Y. Hwangbo, J. I. Mullins, and L. Corey. 2002. Evidence for human immunodeficiency virus type 1 replication in vivo in CD14(+) monocytes and its potential role as a source of virus in patients on highly active antiretroviral therapy. J Virol. 76:707-716.
5. Qi, X., Y. Koya, T. Saitoh, Y. Saitoh, S. Shimizu, K. Ohba, N. Yamamoto, S. Yamaoka, and N. Yamamoto. 2007. Efficient induction of HIV-1 replication in latently infected cells through contact with CD4+ T cells: involvement of NF-kappaB activation. Virology. 361:325-334.
6. Crowe, S. M., J. Mills, T. Elbeik, J. D. Lifson, J. Kosek, J. A. Marshall, E. G. Engleman, and M. S. McGrath. 1992. Human immunodeficiency virus-infected monocyte-derived macrophages express surface gp120 and fuse with CD4 lymphoid cells in vitro: a possible mechanism of T lymphocyte depletion in vivo. Clin Immunol Immunopathol. 65:143-151.
7. Mikovits, J. A., N. C. Lohrey, R. Schulof, J. Courtless, and F. W. Ruscetti. 1992. Activation of infectious virus from latent human immunodeficiency virus infection of monocytes in vivo. J Clin Invest. 90:1486-1491.
8. Greco, G., S. Pal, R. Pasqualini, and L. M. Schnapp. 2002. Matrix fibronectin increases HIV stability and infectivity. J Immunol. 168:5722-5729.
9. Takenouchi, T., S. Sugama, Y. Iwamaru, M. Hashimoto, and H. Kitani. 2009. Modulation of the ATP-lnduced release and processing of IL-1beta in microglial cells. Crit Rev Immunol. 29:335-345.
10. Kyei, G. B., C. Dinkins, A. S. Davis, E. Roberts, S. B. Singh, C. Dong, L. Wu, E. Kominami, T. Ueno, A. Yamamoto, M. Federico, A. Panganiban, I. Vergne, and V. Deretic. 2009. Autophagy pathway intersects with HIV-1 biosynthesis and regulates viral yields in macrophages. J Cell Biol. 186:255-268.
11. Shattock, R. J., J. S. Friedland, and G. E. Griffin. 1993. Release of human immunodeficiency virus by THP-1 cells and human macrophages is regulated by cellular adherence and activation. J Virol. 67:3569-3575.
12. Cecilia, D., V. N. KewalRamani, J. O'Leary, B. Volsky, P. Nyambi, S. Burda, S. Xu, D. R. Littman, and S. Zolla-Pazner. 1998. Neutralization profiles of primary human immunodeficiency virus type 1 isolates in the context of coreceptor usage. J Virol. 72:6988-6996.
13. Pert, C. B., J. M. Hill, M. R. Ruff, R. M. Berman, W. G. Robey, L. O. Arthur, F. W. Ruscetti, and W. L. Farrar. 1986. Octapeptides deduced from the neuropeptide receptor-like pattern of antigen T4 in brain potently inhibit human immunodeficiency virus receptor binding and T-cell infectivity. Proc Natl Acad Sci USA. 83:9254-9258.
14. Dong, C., C. Kwas, and L. Wu. 2009. Transcriptional restriction of human immunodeficiency virus type 1 gene expression in undifferentiated primary monocytes. J Virol. 83:3518-3527.
15. Kilareski, E. M., S. Shah, M. R. Normemacher, and B. Wigdahl. 2009. Regulation of HIV-1 transcription in cells of the monocyte-macrophage lineage. Retrovirology. 6:118.
16. Meanwell, N. A., and J. F. Kadow. 2007. Maraviroc, a chemokine CCR5 receptor antagonist for the treatment of HIV infection and AIDS. Curr Opin Investig Drugs. 8:669-681.
17. Scozzafava, A., A. Mastrolorenzo, and C. T. Supuran. 2002. Non-peptidic chemokine receptors antagonists as emerging anti-HIV agents. J Enzyme Inhib Med Chem. 17:69-76.

What is claimed is:

1. A method of detecting an active HIV infection, useful for minimizing false negatives, comprising:
obtaining, from a patient suspected of having an HIV infection, a sample consisting essentially of cells from a clinically relevant HIV reservoir in the patients body,
placing said sample in an assay vessel,
adding an appropriate aqueous based buffer,
adding an agent to promote sample cell adherence to a solid phase matrix thereby promoting HIV stability and infectivity in said sample cells,
incubating for an appropriate amount of time to allow sufficient sample cell adherence and sufficient syncytia formation to promote HIV stability and infectivity,
washing to remove non-adherent cells,
adding an agent to promote release of virus from within the sample cells,
adding reporter cells engineered to produce a detectable signal when said reporter cells are infected by HIV
wherein detection of said signal is indicative of an active HIV infection in the patient.

2. The method as defined in claim 1 wherein said sample is selected from a tissue sample or a cell sample.

3. The method as defined in claim 1 wherein said clinically relevant HIV reservoir is peripheral blood monocytic cells.

4. The method as defined in claim 3 wherein said peripheral blood monocytic cells are depleted of CD8 lymphocytes.

5. The method as defined in claim 1 wherein said agent to promote sample cell adherence is fibronectin.

6. The method as defined in claim 1 wherein said agent to promote release of virus from within sample cells is ATP.

7. The method as defined in claim 1 wherein said reporter cells contain HIV gene promoter regions fused to reporter genes.

8. The method as defined in claim 7 wherein said reporter cells are selected from the group consisting of: TMZ-bl-luc cells, MaRBLE-luc and GHOST cells.

9. The method as defined in claim 1 wherein said appropriate incubation time is at least about three hours.

10. The method as defined in claim 1 wherein said appropriate incubation time is a range from about three hours to about twenty-four hours.

11. The method as defined in claim 1 wherein said appropriate incubation time is about three hours.

12. The method as defined in claim 1 wherein said assay vessel is a microtiter plate.

13. The method as defined in claim 1 wherein said assay can provide quantitative information on viral burden by using differing amounts of patient cells.

14. A method of detecting drug resistance comprising the method as defined in claim 1, wherein the samples are obtained from patients suspected of having a drug resistant HIV infection and further comprising:
adding a drug known to treat HIV infection,
obtaining a ratio of signal produced in the presence of the drug with the signal produced in the absence of the drug,
wherein a significant increase in the ratio is indicative of an increase in drug resistance.

15. A method of determining drug efficacy comprising the method as defined in claim 1, wherein the samples are obtained from HIV positive patients and further comprising:
adding a drug known to inhibit HIV,
measuring the signal in the presence of the drug relative to the signal in the absence of the drug,
wherein a significant reduction of the signal level is indicative of drug efficacy.

16. A method of determining co-receptor usage/viral receptor tropism comprising the method as defined in claim 1, wherein the samples are obtained from HIV positive patients and further comprising:
adding a drug known to act by blocking HIV binding to a specific cognate entry receptor,
obtaining a ratio of signals produced in the presence of the specifically acting drug with the signal produced in the absence of the drug,
wherein a significant decrease in the ratio is indicative of use by the HIV of the cognate entry receptor.

17. A method of determining drug resistance phenotype comprising the method as defined in claim 1, wherein the samples are obtained from HIV positive patients being treated with at least one drug known to act by blocking HIV binding to a specific cognate entry receptor and further comprising:
obtaining signals from patients at a plurality of time points,
monitoring the signal level over time,
wherein an significant increase in signal level is indicative of the development of resistance to drugs acting by way of blocking said specific cognate entry receptor.

18. A method of detecting changes in co-receptor usage/viral receptor tropism comprising the method as defined in claim 1, wherein the samples are obtained from HIV positive patients and further comprising:
obtaining signals from patients at a plurality of time points,
comparing signals from said plurality of time points,
wherein a significant difference in signal levels is indicative of HIV receptor evolution.

19. The method as defined in claim 1 wherein said washing to remove non adherent cells is omitted.
